(19) United States
(12) Patent Application Publication
(10) Pub. No.: US 2023/0190184 A1
(43) Pub. Date: Jun. 22, 2023

(54) Central Apnea Detection

(71) Applicant: Medical Informatics Corp., Houston, TX (US)

(72) Inventors: Jamie L. S. Waugh, Houston, TX (US); Raajen J Patel, Houston, TX (US); Craig Rusin, Houston, TX (US); Anuj Amit Saheba, Houston, TX (US)

(73) Assignee: Medical Informatics Corp., Houston, TX (US)

(21) Appl. No.: 18/069,061

(22) Filed: Dec. 20, 2022

Related U.S. Application Data
(60) Provisional application No. 63/265,817, filed on Dec. 21, 2021.

(51) Int. Cl. A61B 5/00; A61B 5/0205

(52) U.S. Cl. CPC A61B 5/4818; A61B 5/0205; A61B 5/7282

(57) ABSTRACT

Existing apnea monitors fail to detect clinically important apnea events because they fail to distinguish cardiac artifacts from a chest impedance signal and therefore fail to detect cessation of breathing. A system and method are disclosed that provides improved apnea detection, particularly in a neonatal setting. The disclosed techniques filter out cardiac artifacts from the chest impedance signal, allowing determining a probability of an apnea event. Detection of an apnea event may then be used to trigger an alarm, initiate automatic physical stimulation of the patient, or both.

### CROSS-REFERENCE TO RELATED APPLICATION

This Patent Application claims the benefit of U.S. Provisional Pat. Application No. 63/265,817 filed on Dec. 21, 2021, entitled “Central Apnea Detection.” The disclosure of the prior application is considered part of and is incorporated by reference into this Patent Application.

### TECHNICAL FIELD

The present invention relates to the field of detection of apnea, and in particular to the detection of central apnea.

### BACKGROUND ART

Apnea is the temporary cessation of breathing. Apnea can occur at any stage of development and is particularly common in premature infants, where it may be called apnea of prematurity (AOP). AOP is quite different from adult sleep apnea. While AOP is a developmental disorder, the reasons behind the propensity for apnea in immature infants are not entirely clear. Although the pathogenesis of AOP is poorly understood, the immature pulmonary reflexes and breathing responses to hypoxia and hypercapnia likely contribute to the occurrence or severity of AOP. It may also be exacerbated by coexisting factors or disease states.

AOP is an important and common clinical problem and is often the rate-limiting process in Neonatal Intensive Care Unit (NICU) discharge. Accurate detection of episodes of clinically important neonatal apnea using existing chest impedance monitoring is a clinical imperative.

Apneas are serious clinical events that need immediate medical attention. However, existing monitors for apnea, particularly for neonatal infants, are unsatisfactory-they miss many serious events. For this reason, they often fail to recognize apnea and therefore fail to provide a warning signal to NICU personnel alerting them to the fact that the infant is not breathing.

Thus, there is a need in the art for techniques that provide improved detection of apneas.

### BRIEF DESCRIPTION OF DRAWINGS

The accompanying drawings, which are incorporated in and constitute a part of this specification, illustrate an implementation of apparatus and methods consistent with the present invention and, together with the detailed description, serve to explain advantages and principles consistent with the invention. In the drawings,

FIG. 1 is a screenshot illustrating a graphical user interface for displaying an indication of an apnea event according to one embodiment.

FIG. 2 is a screenshot illustrating a graphical user interface for displaying an indication of an apnea event according to one embodiment.

FIG. 3 is a flowchart illustrating a technique for detecting an apnea event according to one embodiment.

FIG. 4 is a block diagram illustrating a system for collecting, archiving, and processing arbitrary data in a healthcare environment according to one embodiment.

FIG. 5 is a block diagram illustrating a computer server for use in the system of FIG. 4.

FIG. 6 is a screenshot illustrating a graphical user interface for tracking apnea events according to one embodiment.

### DETAILED DESCRIPTION

In the following description, for purposes of explanation, numerous specific details are set forth in order to provide a thorough understanding of the invention. It will be apparent, however, to one skilled in the art that the invention may be practiced without these specific details. In other instances, structure and devices are shown in block diagram form to avoid obscuring the invention. References to numbers without subscripts are understood to reference all instances of subscripts corresponding to the referenced number. Moreover, the language used in this disclosure has been principally selected for readability and instructional purposes, and may not have been selected to delineate or circumscribe the inventive subject matter, resort to the claims being necessary to determine such inventive subject matter. Reference in the specification to “one embodiment” or to “an embodiment” means that a particular feature, structure, or characteristic described in connection with the embodiments is included in at least one embodiment of the invention, and multiple references to “one embodiment” or “an embodiment” should not be understood as necessarily all referring to the same embodiment.

Although some of the following description is written in terms that relate to software or firmware, embodiments can implement the features and functionality described herein in software, firmware, or hardware as desired, including any combination of software, firmware, and hardware. References to daemons, drivers, engines, modules, or routines should not be considered as suggesting a limitation of the embodiment to any type of implementation.

Apnea is a serious clinical event that needs medical attention within seconds and is very common in premature infants. Newborns tend to have an irregular breathing pattern and stop breathing for a few seconds at a time. Physicians differ in how they determine which events are of clinical importance. A common rule of thumb considers apnea to be a clinical event if (a) cessation of breathing lasts for more than 20 seconds or (b) cessation of breathing lasts for more than 10 seconds and is accompanied by either bradycardia (slowing of the heart) or oxygen desaturation. In neonates, bradycardia is typically defined as a heart rate less than 100 beats per minute and oxygen desaturation is typically defined as an SpO₂ less than 80%. Bradycardia and desaturation events can occur without a cessation of breathing. When these vital thresholds are passed within a defined period of the start or end of a neonate’s significant cessation of breathing event, further deterioration has likely occurred.

Clinical apneas occur in more than half of babies whose birth weight is less than 1500 g, and in almost all infants whose birth weight is less than 1000 g. Apnea may be a cause or an effect of many other clinical problems, such as hypoxemia, hypoglycemia, neurological injury, or sepsis. Apnea is also a common manifestation of an immature neurological system in babies who were born very preterm but otherwise have no clinical pathology. Three types of apnea are common in premature infants: obstructive apnea, central apnea, and mixed apnea. Obstructive apnea is a blockage of the airway, typically accompanied by struggling or thrashing movements of the infant. Central apnea is the cessation of respiratory drive, and the infant makes no effort to breathe and usually remains very still. Mixed apneas typically begin with an obstructive event, and then change to central apnea. On the other hand, there is evidence that central apnea can also become obstructive apnea, so some sources suggest that the distinction between the two types should not be held too rigidly. In many cases, the apnea is combined with, or induces, bradycardia (a significant slowing of the heart rate). When accompanied by bradycardia, oxygen desaturation, or both, these apneas are serious clinical events that need immediate medical attention and investigation for associated pathology. Moreover, central apnea is taken to indicate immaturity of control of respiration, and discharge from NICUs is typically delayed until apneas have been absent for 3 to 8 days. These regulations exist so that infants can be safely discharged by reducing the likelihood that they will have another apnea episode once they are at home, without monitoring devices and clinicians.

Bedside monitors attempt to detect apnea based on continuous monitoring of chest impedance (CI) physiological data. Using electrodes that are also used to monitor the electrocardiogram (ECG), a small high-frequency (e.g., 52.6 kHz) voltage is applied to the chest, and the resulting high-frequency current is measured. The measured impedance Z is equal to the applied voltage V divided by the observed current. The measured impedance is typically in the range of 50 to 300 ohms and is related to the conductivity of muscle, skin, other tissues, and the contacts between electrodes and skin. When the infant is breathing, the impedance fluctuates with each breath. In a central apneic event, the chest impedance changes are reduced because of the cessation of breathing. However, the beating of the heart also causes fluctuations in impedance as every heartbeat pumps blood out of the thorax. Thus, even during an apneic event, the chest impedance fluctuates and can reach thresholds for detection as breaths, foiling the monitor’s apnea alarm.

Detection of cessation of breathing in neonates is particularly difficult because a neonate’s more malleable rib cage moves as its heart beats. Chest impedance leads may mistake the resulting change in rib cage diameter for respiration. Current apnea detectors do not address this problem so in these cases, clinicians must rely on alarms that indicate subsequent deterioration of the patient such as bradycardia.

As a result of these cardiac artifacts, some episodes of central neonatal apnea are missed altogether, and some of those episodes are severe. Some studies have found that conventional apnea monitors miss over 10% of even extreme apnea events because of cardiac artifacts in the chest impedance signal.

The techniques described below remove these cardiac artifacts from the chest impedance waveform used to measure respiration. Although described below in terms of real-time physiological data, the same techniques may be used on historical physiological data for retrospective analysis.

Due to the increased detection of apnea events, in certain embodiments, automated interaction may be utilized to stimulate the premature infant during an apnea event.

The disclosure below describes techniques for improving the detection of central apnea. More specifically, the standard CI signal used to monitor respiration rate is analyzed to filter out the contribution to the chest impedance signal that arises from a beating heart, and an indication of a breathing cessation event is indicated.

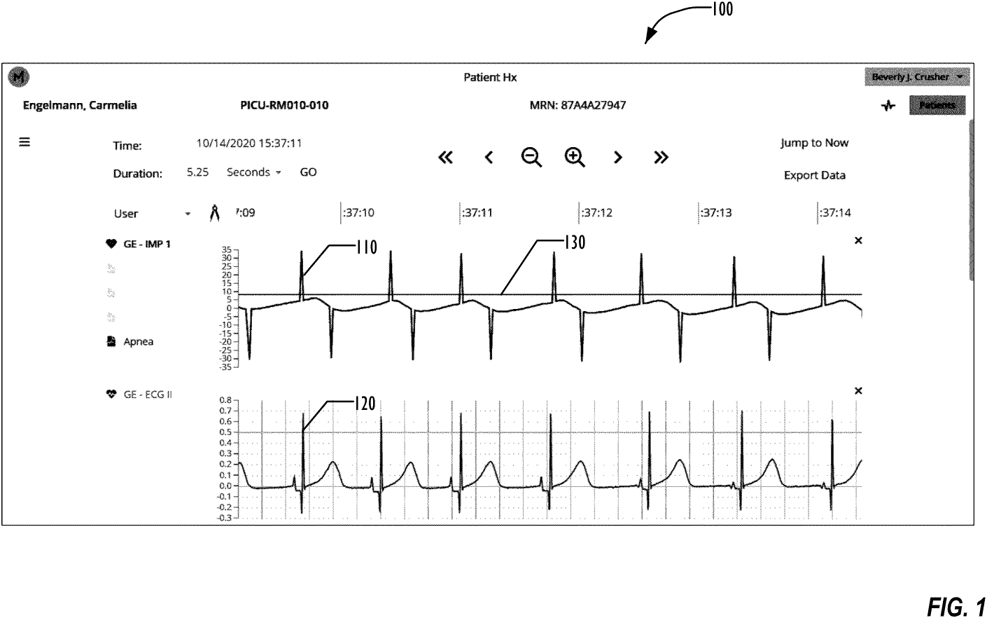

FIG. 1 is a screenshot illustrating a graphical user interface 100 according to one embodiment in which an indication of central apnea is displayed. Two waveform lanes are illustrated in the graphical user interface 100: a CI waveform 110 and an ECG waveform 120. The example waveform data in FIG. 1 is not intended to be medically accurate. In this example, the patient is experiencing an apnea condition, indicated by line 130, even though the CI waveform 110 is not flat. The CI waveform is experiencing fluctuations as a result of the heartbeat illustrated in the ECG waveform 120. We know this because both waveforms repeat cyclically at the same rate. In some embodiments, one or both of an audible alarm and a visual alarm indication may be generated in response to a determination that an apnea condition exists, in addition to the indication shown in line 130. In other embodiments, an automatic response may be triggered to physically stimulate the infant, which may cause cessation of the apneic event.

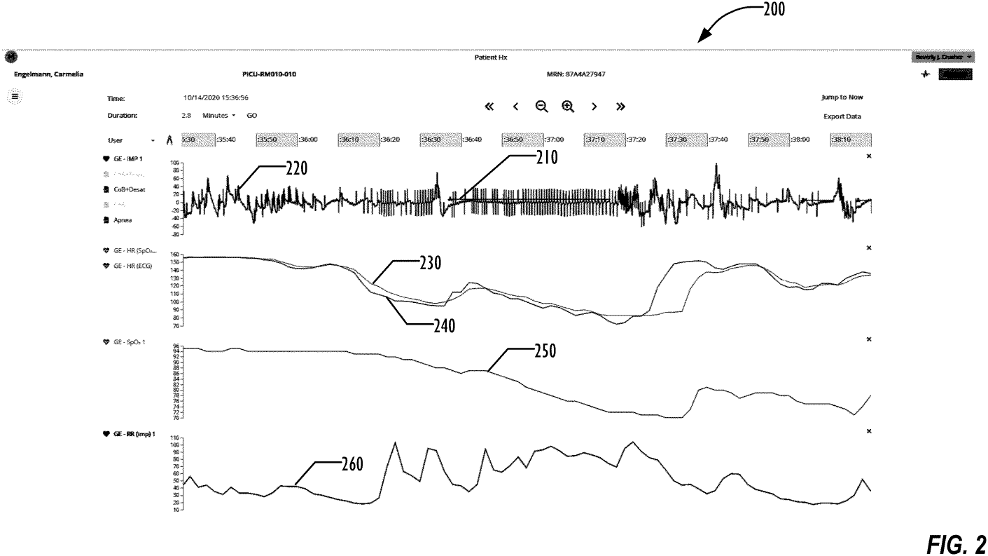

FIG. 2 is a screenshot illustrating a graphical user interface 200 according to one embodiment similar to the graphical user interface 100. In this example, graph 220 is an example CI waveform, graph 230 indicates a heart rate as determined from photoplethysmogram data, graph 240 indicates a heart rate as determined from ECG data, graph 250 indicates SpO₂ data determined from photoplethysmogram data, and graph 260 indicates a calculated respiration rate based solely on the CI waveform. In this example, an apnea event is shown as indicated by horizontal line 210, indicating that the patient ceased breathing and met bradycardia and oxygen desaturation criteria. In one implementation, apnea events are indicated as beginning only after a threshold time (e.g., 20 seconds) during which a cessation of breathing was identified and bradycardia and oxygen desaturation occurred. This reduces nuisance alarm indications for short apnea events that terminate without intervention.

While both FIG. 1 and FIG. 2 illustrate a real-time display of information showing the presence of an apnea event, some embodiments, in which real-time data is archived for research or clinical purposes may provide the ability to investigate historical patient data, analyze that data, and indicate the presence of apnea events in the historical data. Such historical investigation may be useful to detect prior apneas that were unnoticed and provide clinicians with better information to evaluate the health of the patient.

For clarity, the description below focuses on cessation of breathing events as being apnea events. However, different medical personnel may define apnea events as a cessation of breathing without other factors or a cessation of breathing combined with one or more other factors such as bradycardia or oxygen desaturation. The system may be configured for any combination of those additional factors as desired. For example, a patient may cease breathing for more than a threshold time, but maintain a normal heart rate and oxygen saturation, in which case some configurations of the system described below may not indicate an apnea event. Embodiments of the system described below may allow clinical personnel to select what combination of criteria are to be used for determining an apnea event.

Inputs to the central apnea detector may include a CI waveform, an ECG waveform, and heart rate and oxygen saturation (SpO₂) vitals. Each of the signals may undergo cleaning to eliminate data artifacts if desired.

The cardiac artifact component of the chest impedance can be identified based on its shared phase (progression of frequency) with any signal that captures heart movement, (e.g., an ECG signal), as described below. The first step is to model the progression of the cardiac phase. This can be done in multiple ways, using any signal that captures heart movement. For example, the cardiac phase could be calculated by taking the phase of the Hilbert transform applied to the photoplethysmogram waveform.

In one implementation, to determine the cardiac phase standard peak/trough detection is performed to find the R peaks in the ECG signal that occur each cardiac cycle. A cardiac phase signal ϕ is made to model the heart’s frequency at any given time by setting the phase to 0 at each R peak time and having the phase increase linearly with time to 2π = 0 radians for samples in between each set of consecutive R peaks. Thus, the cardiac phase signal ϕ for a segment of data that contains N samples is an Nx1 vector

$$ϕ = \left[\begin{matrix}{ϕ}_{1} \\ {ϕ}_{2} \\ ⋮ \\ {ϕ}_{N}\end{matrix}\right]$$

Components of the measured chest impedance signal CIᵢₙᵢₜ that correspond to the cardiac phase (i.e., the cardiac artifact) may then be extracted using a Fourier series approximation of CIᵢₙᵢₜ that is a function of the determined cardiac phase. Although the description below is written in terms of Fourier series, other frequency or time domain analysis and approximation techniques may be used.

The Fourier series approximation uses M harmonics stored as a 1xM vector

$$M = \left[\begin{matrix}1 & 2 & ⋯ & M\end{matrix}\right]$$

In some embodiments, M=3 sufficiently captures the frequency components contained in the cleaned CI, but other numbers of harmonics may be used as desired.

This leads us to an NxM cardiac phase harmonic matrix ϕH, where each column represents the cardiac phase progression of a particular harmonic of the Fourier series representation of the cardiac artifact:

$$ϕ H = \left[\begin{matrix}{ϕ}_{1} & 2 {ϕ}_{1} & ⋯ & M {ϕ}_{1} \\ {ϕ}_{2} & 2 {ϕ}_{2} & ⋯ & M {ϕ}_{2} \\ ⋮ & ⋮ & ⋱ & ⋮ \\ {ϕ}_{N} & 2 {ϕ}_{N} & ⋯ & M {ϕ}_{N}\end{matrix}\right]$$

The frequency components F of the cardiac artifact Fourier series approximation can be calculated from the cardiac phase, resulting in an Nx2M matrix:

$$F = \left[\begin{matrix}cos \left(ϕ H\right) & sin \left(ϕ H\right)\end{matrix}\right]$$

$$= \left[\begin{matrix}cos \left(ϕ\right) & cos \left(2 ϕ\right) & ⋯ & cos \left(M ϕ\right) & sin \left(ϕ\right) & ⋯ & sin \left(M ϕ\right)\end{matrix}\right]$$

$$= \left[\begin{matrix}c o s \left({ϕ}_{1}\right) & c o s \left(2 {ϕ}_{1}\right) & ⋯ & c o s \left(M {ϕ}_{1}\right) & s i n \left({ϕ}_{1}\right) & ⋯ & s i n \left({ϕ}_{1}\right) \\ c o s \left({ϕ}_{2}\right) & c o s \left(2 {ϕ}_{2}\right) & ⋯ & c o s \left(M {ϕ}_{2}\right) & s i n \left({ϕ}_{2}\right) & ⋯ & s i n \left(M {ϕ}_{2}\right) \\ ⋮ & ⋮ & ⋱ & ⋮ & ⋮ & ⋱ & ⋮ \\ c o s \left({ϕ}_{N}\right) & c o s \left(2 {ϕ}_{N}\right) & ⋯ & c o s \left(M {ϕ}_{N}\right) & s i n \left({ϕ}_{N}\right) & ⋯ & s i n \left(M {ϕ}_{N}\right)\end{matrix}\right]$$

We may then solve for the cardiac artifact Fourier series coefficients using the above cardiac artifact frequency components and the chest impedance signal. The Fourier series coefficients can be represented as a 2Mx1 vector:

$$\left[\begin{matrix}α \\ β\end{matrix}\right] = \left[\begin{matrix}{α}_{1} \\ {α}_{2} \\ ⋮ \\ {α}_{M} \\ {β}_{1} \\ {β}_{2} \\ ⋮ \\ {β}_{M}\end{matrix}\right]$$

The cardiac artifact Fourier series coefficients are calculated as follows:

$$\left[\begin{matrix}α \\ β\end{matrix}\right] = {F}^{- 1} C {I}_{i n i t}$$

The cardiac artifact CIₐᵣₜ approximation may then be calculated as follows:

$$\begin{matrix}C {I}_{a r t} = \text{F} \left[\begin{matrix}α \\ β\end{matrix}\right] \\ = {\sum }_{m = 1}^{M} \left[{α}_{m} cos \left(m ϕ\right) + {β}_{m} sin \left(m ϕ\right)\right]\end{matrix}$$

$$= \left[\begin{matrix}{\sum }_{m = 1}^{M} \left[{α}_{m} cos \left(m {ϕ}_{1}\right) + {β}_{m} sin \left(m {ϕ}_{1}\right)\right] \\ {\sum }_{m = 1}^{M} \left[{α}_{m} cos \left(m {ϕ}_{2}\right) + {β}_{m} sin \left(m {ϕ}_{2}\right)\right] \\ ⋮ \\ {\sum }_{m = 1}^{M} \left[{α}_{m} cos \left(m {ϕ}_{N}\right) + {β}_{m} sin \left(m {ϕ}_{N}\right)\right]\end{matrix}\right]$$

The cardiac artifact CIₐᵣₜ may then be removed from the measured chest impedance signal CIᵢₙᵢₜ, producing a filtered chest impedance signal CI_{filt}

$$C {I}_{f i l t} = C {I}_{i n i t} - C {I}_{a r t}$$

Once the cardiac artifact has been removed from the chest impedance, standard signal processing techniques of the filtered chest impedance and thresholding of the heart rate and oxygen saturation vitals can be used to identify cessation of breathing, bradycardia, and desaturation events. When there is little lung movement (small standard deviation) in the filtered chest impedance signal, the neonate is considered not breathing and a cessation of breathing event is identified.

An apnea event probability may be estimated using a Fermi function such as the following:

$${P}_{f i t} \left(σ\right) = \frac{1}{1 + {e}^{\left(12 \left(σ - 0.44\right)\right)}}$$

where σ is the standard deviation of Cl_{filt}. The parameters of the above Fermi function are illustrative and by way of example only. Other Fermi functions or types of functions may be used to calculate the probability of an apnea based on CI_{filt}. In some embodiments, when the apnea event probability exceeds a predetermined threshold value, the system is triggered and displays an alarm indication of an apnea event in a graphical user interface such as the ones illustrated in FIGS. 1 and 2. Furthermore, a set of alarm criteria may be developed based on the relative apnea event probability over a defined period. In addition, in embodiments where one or more of bradycardia and desaturation are considered apnea criteria, the result of measurement of heart rate and SpO₂ data may be combined with the probability calculated above to trigger the alarm indication.

When these vital thresholds are passed within a defined period of the start or end of a cessation of breathing event, a cessation of breathing with bradycardia and desaturation event is identified. The techniques described herein may be used for determining apneic events in children and adults, although the specific criteria such as heart rate and oxygen desaturation may be different in non-neonate groups.

The resulting improved system for detecting apnea events avoids missing apnea events that are masked by patient heartbeats in existing apnea detection systems.

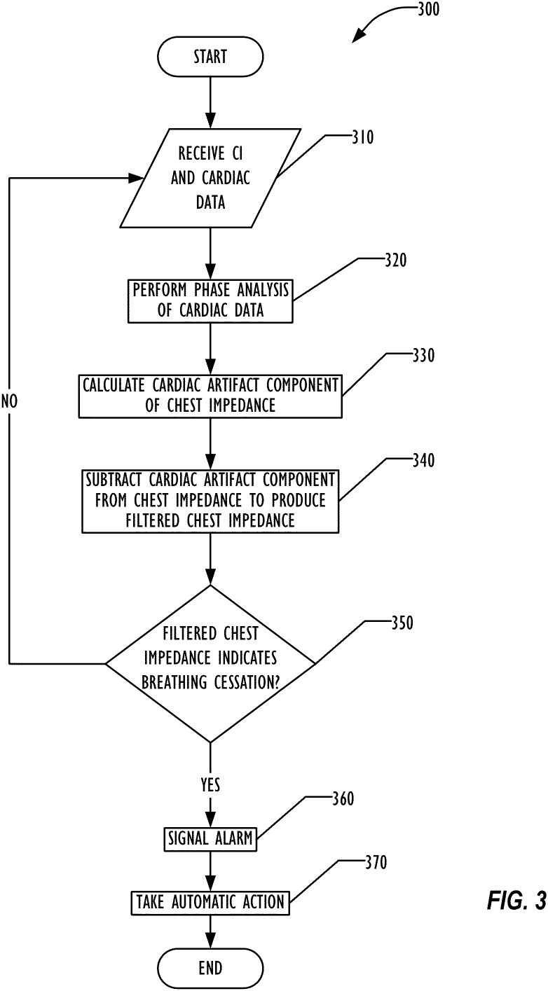

FIG. 3 is a flowchart illustrating a technique 300 for detecting and automatically acting on an apnea event according to one embodiment. In block 310, the system receives real-time CI and cardiac data from sensors attached to a patient. A phase analysis of the cardiac data (e.g., ECG) is performed in block 320 as described above to create a phase signal ϕ and cardiac artifact phase harmonic matrix ΦH. In block 330, using the algorithm described above, the cardiac artifact component of the chest impedance CIₐᵣₜ is calculated. Then in block 340, the filtered chest impedance Ci_{filt}is calculated by subtracting the cardiac artifact component CIₐᵣₜ from the measured chest impedance CIᵢₙᵢₜ. In block 350 if the filtered chest impedance CI_{filt} indicates breathing cessation an alarm may be triggered in the graphical user interface such as those illustrated in FIGS. 1-2, as an audible alarm, or in any other way known in the art for generating alarms. In some embodiments, a breathing cessation may be detected if the probability of an apnea event calculated as described above, reaches or exceeds a predefined threshold value or meets a set of criteria over a predefined duration. In some implementations, the overall periods in which the threshold is met are evaluated, rather than using a strict time threshold. For example, if there are two 12-second periods in which the threshold is met separated by one second, those two segments may be combined into one cessation of breathing event spanning 25 seconds.

If no breathing cessation is detected in block 350, the technique continues receiving CI and cardiac data in block 310. If a cessation of breathing is detected in block 350, an alarm is signaled in block 360. In block 370, where available, an automatic action may be taken to physically stimulate the patient responsive to the apnea event, such as creating a vibration, blowing air, or initiating another physical stimulus that may cause the patient to restart breathing. Where the system implementing technique 300 is configured to use one or more of bradycardia and desaturation as part of the threshold evaluation of an apnea event, technique 300 may include other actions not illustrated in FIG. 3 for detecting bradycardia and desaturation from heart rate and SpO₂ data received from patient monitors. Where no automatic physical stimulation of the patient is available, a clinical staff member may approach the patient to provide tactile stimulation manually, such as a gentle tap to the sole of the foot or rubbing the back. In some cases, where physical stimulation does not initiate spontaneous respiration, an apnea event may require additional interventions, such as ventilation with oxygen.

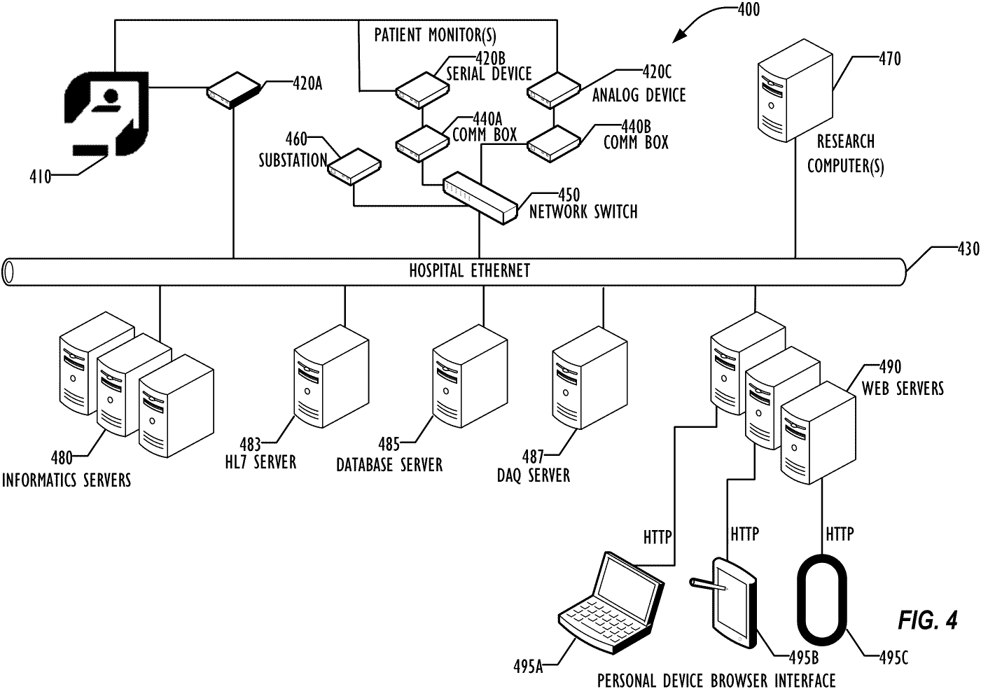

FIG. 4 is a block diagram illustrating a system 400 for collecting, archiving, and processing arbitrary data in a healthcare environment according to one embodiment. The system 400 is described in more detail in U.S. Pat. No. 10,8892,045, “Distributed Grid-Computing Platform for Collecting, Archiving, and Processing Arbitrary Data in a Healthcare Environment,” which is incorporated by reference in its entirety for all purposes.

As illustrated, there are five types of servers: the data acquisition (DAQ) server 487, the informatics server(s) 480, the database server 485, the HL7 server 483, and the web server (s) 490. Any number of any of the types of servers may be deployed as desired. All of the servers 480-490 connect to each other and the bedside monitors via one or more hospital networks 430. Although illustrated as a single hospital Ethernet network 430, any number of interconnected networks may be used, using any desired networking protocols and techniques.

Also connected to the hospital network 430 are a number of bedside monitors for monitoring physiological data of a patient in bed 410. These bedside monitors may include network-connected monitors 420A, which can deliver digital physiological data to the hospital network 430, serial devices 420B, which produce digital data but are not directly connected to a network, and analog devices 420C, which produce analog data and are not directly connected to a network. Communication boxes 440A and 440B allow connecting the serial devices 420B and analog devices 420C, respectively, to the hospital network 430, typically through a network switch 450. In addition, a substation 460 may also be connected to network 430 via the network switch 450 for performing data manipulation and time synchronization as described below. Any number of bedside monitors 420 may be used as determined advisable by physicians and other clinical staff for the patient in bed 410.

Although FIG. 4 illustrates the bedside monitors and associated communication devices connected directly or indirectly to the hospital network 430, remote bedside monitoring devices may be used as part of the system 400, such as home monitoring devices, connected to the hospital network 430 indirectly through the Internet or other communication techniques.

Additionally, one or more research computers 470 may be connected, directly or indirectly, to the hospital network 430, allowing researchers to access aggregated data collected from bedside monitors 420 for performing analytics and development.

The webservers 490 are configured for communicating with personal devices such as laptop 495A, tablet 495B, or smartphone 495C via a web browser interface using HyperText Transport Protocol (HTTP).

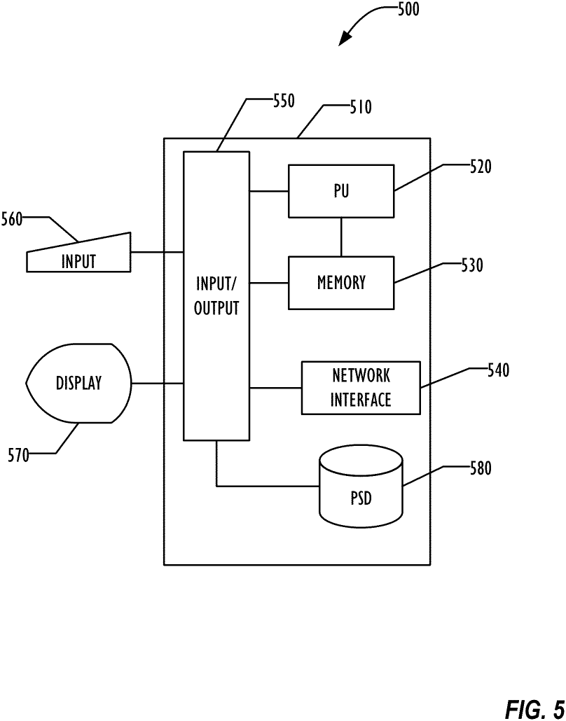

Referring now to FIG. 5, an example computer 500 for use as one of the servers 480-490 is illustrated in block diagram form. Example computer 500 comprises a system unit 510 which may be optionally connected to an input device or system 560 (e.g., keyboard, mouse, touch screen, etc.) and display 570. A program storage device (PSD) 580 (sometimes referred to as a hard disc) is included with the system unit 510. Also included with system unit 510 is a network interface 540 for communication via a network with other computing and corporate infrastructure devices (not shown). Network interface 540 may be included within system unit 510 or be external to system unit 510. In either case, system unit 510 will be communicatively coupled to network interface 540. Program storage device 580 represents any medium of non-volatile storage including, but not limited to, all forms of optical and magnetic, including solid-state, storage elements, including removable media, and may be included within system unit 510 or be external to system unit 510. Program storage device 580 may be used for storage of software that when executed controls system unit 510, data for use by the computer 500, or both.

System unit 510 may be programmed to perform methods in accordance with this disclosure (an example of which is in FIG. 3). System unit 510 comprises a processor unit (PU) 520, input-output (I/O) interface 550, and memory 530. Processor unit 520 may include any programmable controller device, such as microprocessors available from Intel Corp. and other manufacturers. Memory 530 may include one or more memory modules and comprise random access memory (RAM), read-only memory (ROM), programmable read-only memory (PROM), programmable read-write memory, and solid-state memory. One of ordinary skill in the art will also recognize that PU 520 may also include some internal memory including, for example, cache memory.

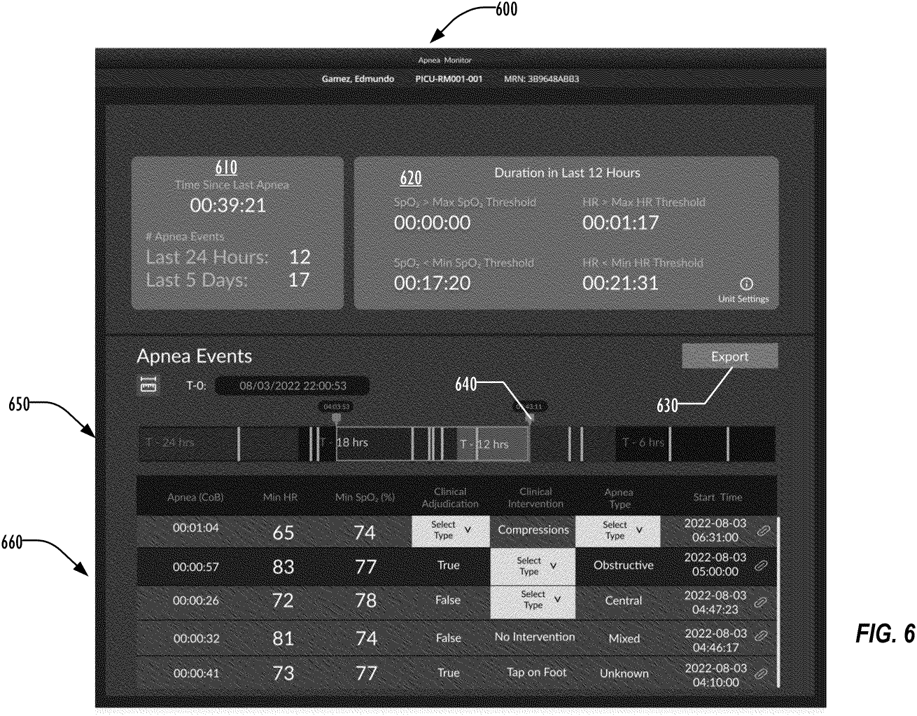

FIG. 6 is a screenshot illustrating an example graphical user interface 600 for monitoring historical information about apnea events for a patient according to one embodiment. Each apnea event is detected and recorded using the systems and techniques described above and may then be sent to the graphical user interface 600 for display. Aggregate information about the apnea events is also sent to the graphical user interface 600 for display. In this example graphical user interface 600, a first area 610 provides historical information about the number of recent apnea events, in this example a time since the last apnea event and the number of apnea events in the last 24 hours and five days. The display of “last 24 hours” and “last 5 days” is by way of example only, and other useful historical periods could be provided in addition to or instead of the ones illustrated in FIG. 6. A second area 620 provides historical information about the total length of apnea events in the last 12 hours. In this example, two types of oxygen saturation (SpO₂)-related records are displayed: SpO₂ > Max SpO₂ Threshold and SpO₂ < Min. SpO₂ Threshold. Similarly, two types of heart rate (HR)-related records are displayed: HR > Max HR Threshold and HR < Min. HR Threshold. Other types of historical information may be displayed in the second area 620 in addition to or instead of the information illustrated in FIG. 6.

Below the first area 610 and the second area 620 is an area providing historical information about individual apnea events. A timeline 650 in this example provides indicators of apnea events in the period 24 hours up to a selected “time zero.” Other time ranges could be displayed if desired. A pair of calipers 640 may be placed on the timeline to select a particular window for detailed display in area 660 below the timeline 650. An Export button 630 allows exporting information about the selected time window to an electronic medical records (EMR) system.

In the detailed information area 660, details about each apnea event may be displayed. As illustrated in FIG. 6, the information displayed for each event includes the length of the cessation of breathing, a minimum HR occurring within a specified period relative to the event, a minimum oxygen saturation level occurring within a specified period relative to the event, whether a clinician adjudicated the event as detected by the system as a real apnea event, the type of clinical intervention that was performed in response to the apnea event, such as compressions, a tap on a foot, or no intervention, a designation of the type of apnea event, such as obstructive, central, mixed, or unknown, and a start time of the event. Other detailed information may be provided as desired in the user interface 600.

The areas and arrangement of areas in the example graphical user interface 600 of FIG. 6 are illustrative and by way of example only, and other areas and arrangements of areas may be provided as desired. Although some of the elements of the graphical user interface are illustrated as drop-down type user interaction elements, other types of user interaction elements may be used as desired. Color may be used to improve the user experience. For example, in some implementations, the indicator in timeline 650 of a particular apnea event may be displayed in a different color than the other indicators, while the corresponding row in the details area 660 is highlighted or indicated in a different color.

In clinical settings without the disclosed patient monitoring system, nurses or other clinical personnel manually record apnea events that occur on their shift in a flow sheet, either by handwriting data on a piece of paper or by typing data into an electronic form. In either event, the flow sheet requires clinical attention and is subject to human transcription error. The disclosed system allows automatically populating clinical flow sheets with apnea event information, which can significantly reduce both the manual labor of the nurse or other clinical personnel, as well as reducing the possibility of human error in recording the information. This may include the length of cessation of breathing, the start time, and SpO₂ and HR information. Other items, such as clinical adjudication, type of apnea, and the type of clinical intervention, might still be entered by the nurse, but would be entered in a standardized form by selecting from a list of possible entries. For example, clinical personnel report that detecting the end of an apnea event and thus calculating the length of the apnea event can be difficult. By automatically detecting both the beginning and end of the apnea event, the disclosed system may automatically calculate the length of the event, further reducing the load on clinical personnel and reducing manual data entry errors.

By collecting objective data on apnea events, instead of depending on detection by clinical personnel and improving on existing alarm systems, better tracking of apnea events may be provided. This improved tracking results in fewer false alarms but also in the detection of apnea events not currently detected in clinical settings. One result of such better data may be reduced clinical stays for infants, reducing costs of childcare both for the clinical facility and for the parents, as well as improving the quality of life of both the infant and the parents. Because patients often are not released until a certain amount of apnea-free time passes, reducing false alarms of apnea events can allow patients to be released earlier, for example.

While certain exemplary embodiments have been described in detail and shown in the accompanying drawings, it is to be understood that such embodiments are merely illustrative of and not devised without departing from the basic scope thereof, which is determined by the claims that follow.

We claim:

1. A method of detecting apnea in a patient, comprising:
receiving chest impedance physiological data and cardiac physiological data from bedside monitoring equipment associated with the patient;
modeling cardiac phase using the cardiac physiological data;
calculating a frequency or time domain approximation of a cardiac artifact based on a representation of the cardiac phase and the chest impedance physiological data;
removing the cardiac artifact from the chest impedance physiological data, producing a filtered chest impedance data;
calculating a probability of an apnea event based on the filtered chest impedance data; and
generating an alarm indication responsive to a set of criteria being met based on the probability of the apnea event over a period of time.

2. The method of claim 1, further comprising:
triggering an automatic physical stimulation of the patient responsive to the apnea event.

3. The method of claim 1, further comprising:
calculating a length of the apnea event.

4. The method of claim 1, further comprising:
recording apnea events for the patient;
sending information about historical apnea events to a graphical user interface;
calculating aggregate information about the recorded apnea events for the patient; and
sending the aggregate information about the recorded apnea events to the graphical user interface.

5. The method of claim 4, wherein recording apnea events for the patient comprises recording oxygen saturation and heart rate data associated with the apnea events.

6. The method of claim 1, further comprising receiving heart rate and oxygen saturation measurements from the bedside monitoring equipment,
wherein the set of criteria includes heart rate and oxygen saturation criteria.

7. The method of claim 1, wherein the patient is a neonatal infant.

8. A patient monitoring system for detecting apnea events in a patient, comprising:
bedside monitoring equipment associated with the patient that produces chest impedance physiological data and cardiac physiological data;
a computer system programmed to:
receive the chest impedance physiological data and the cardiac physiological data;
model cardiac phase using the cardiac physiological data;
calculate a frequency or time domain approximation of a cardiac artifact based on a representation of the cardiac phase and the chest impedance physiological data;
remove the cardiac artifact from the chest impedance physiological data, producing a filtered chest impedance data;
calculate a probability of an apnea event based on the filtered chest impedance data; and
generate an alarm indication responsive to a set of criteria being met based on the probability of the apnea event over a period of time.

9. The patient monitoring system of claim 8, wherein the computer system is further programmed to:
trigger an automatic physical stimulation of the patient.

10. The patient monitoring system of claim 8, wherein the computer system is further programmed to:
calculate a length of the apnea event.

11. The patient monitoring system of claim 8, wherein the computer system is further programmed to:
record apnea events for the patient;
send information about historical apnea events to a user interface;
calculate aggregate information about the recorded apnea events for the patient; and
send the aggregate information about the recorded apnea events to the user interface.

12. The patient monitoring system of claim 11, wherein the computer system is further programmed to record oxygen saturation and heart rate data associated with the apnea events.

13. The patient monitoring system of claim 8, wherein the computer system is further programmed to receive heart rate and oxygen saturation measurements from the bedside monitoring equipment,
wherein the set of criteria includes heart rate and oxygen saturation criteria.

14. The patient monitoring system of claim 8, wherein the patient is a neonatal infant.

15. A non-transient medium on which is stored software for detecting apnea events in a patient, comprising software that when executed causes a computer system to:
receive chest impedance physiological data and cardiac physiological data from bedside monitoring equipment associated with the patient;
model cardiac phase using the cardiac physiological data;
calculate a frequency or time domain approximation of a cardiac artifact based on a representation of the cardiac phase and the chest impedance physiological data;
remove the cardiac artifact from the chest impedance physiological data, producing a filtered chest impedance data;
calculate a probability of an apnea event based on the filtered chest impedance data; and
generate an alarm indication responsive to a set of criteria being met based on the probability of the apnea event over a period of time.

16. The non-transient medium of claim 15, wherein the software when executed further causes the computer system to:
trigger an automatic physical stimulation of the patient.

17. The non-transient medium of claim 15, wherein the software when executed further causes the computer system to:
calculate a length of the apnea event.

18. The non-transient medium of claim 15, wherein the software when executed further causes the computer system to:
record apnea events for the patient;
send information about historical apnea events to a user interface;
calculate aggregate information about the recorded apnea events for the patient; and
send the aggregate information about the recorded apnea events to the user interface.

19. The non-transient medium of claim 18, wherein the software when executed further causes the computer system to record SpO₂ and heart rate data associated with the apnea events.

20. The non-transient medium of claim 15, wherein the software when executed further causes the computer system to:
receive heart rate and oxygen saturation measurements from the bedside monitoring equipment,
wherein the set of criteria includes heart rate and oxygen saturation criteria.
